(19) United States
(12) Patent Application Publication
(10) Pub. No.: US 2023/0225696 A1
(43) Pub. Date: Jul. 20, 2023

(54) Methods and Systems for Assessment of Pulmonary Hypertension

(71) Applicant: The Board of Trustees of the Leland Stanford Junior University, Stanford, CA (US)

(72) Inventors: Seraina Dual, Stanford, CA (US); Francois Haddad, Stanford, CA (US); Alison Marsden, Stanford, CA (US); Constance M. Verdonk, Stanford, CA (US); Myriam Amsallem, Stanford, CA (US)

(73) Assignee: The Board of Trustees of the Leland Stanford Junior University, Stanford, CA (US)

(21) Appl. No.: 18/151,381

(22) Filed: Jan. 6, 2023

Related U.S. Application Data
(60) Provisional application No. 63/266,510, filed on Jan. 6, 2022.

(51) Int. Cl. A61B 8/04; A61B 8/08

(52) U.S. Cl. CPC A61B 8/04; A61B 8/488; A61B 8/5207; A61B 8/5223

(57) ABSTRACT

Systems and methods for non-invasively estimating right ventricular pressure are provided. Tricuspid regurgitation echocardiography Doppler signals can be acquired utilizing sonography. Digitization and interpolation of the tricuspid regurgitation Doppler signals can be utilized to estimate tricuspid regurgitation and right ventricular pressure and/or provide a metric for signal quality (quality control) as well as increase confidence in the right ventricular pressure estimates.

### CROSS-REFERENCE TO RELATED APPLICATIONS

This application claims priority to U.S. Provisional Application Ser. No. 63/266,510, entitled “Methods and Systems for Assessment of Pulmonary Hypertension” to Dual et al., filed Jan. 6, 2022, which is incorporated herein by reference in its entirety.

### TECHNOLOGICAL FIELD

The disclosure is generally directed to methods and systems to assess pulmonary hypertension using noninvasive techniques.

### BACKGROUND

Pulmonary hypertension (PH) is a condition in which the pressure within the pulmonary artery is too high. The right ventricle pumps blood to the lungs passing through the pulmonary artery to allow oxygenation. The pressure in this side of the heart and in the artery is normally low-usually much lower than systolic or diastolic blood pressure measured within the radial artery of the arm. High pressure in the pulmonary artery can lead to impairment of blood flow through the lungs, resulting in an increase of right ventricle afterload, and further resulting in right ventricle dysfunction.

The initial symptoms of PH are difficulty breathing and fatigue, which are common to many other medical conditions. The lack of distinguishable symptoms makes PH difficult to diagnose. When PH is suspected to be affecting an individual, the affirmative diagnosis of PH is typically performed via right heart catheterization, which is very intrusive invasive, unpleasant, and comes with a risk of infection. Generally, the catheterization procedure is performed under local anesthesia and sedation and requires insertion of a pressure sensor within a flexible tube into a vein (e.g., femoral vein) that is guided up into the right ventricle and pulmonary artery, where arterial pressure is measured.

A Doppler echocardiogram, which utilizes ultrasound sonography, provides a noninvasive means to assess PH. Doppler echocardiograms, however, can be unreliable and highly dependent on the skill of the operator to accurately determine pulmonary arterial and right ventricular pressure. Thus, these scans are typically utilized as primary diagnostic tool prior to performing pulmonary artery catheterization.

### SUMMARY

Several embodiments are directed to methods and systems of estimating right ventricular pressure. In many embodiments, sonography is utilized to acquire tricuspid regurgitation (TR) echocardiography Doppler signal. In several embodiments, the TR curve is traced and the TR Doppler signal is digitized. In many embodiments, digitization of TR Doppler signal is performed by synchronizing traced TR curves with electrocardiography and normalized to the RR interval. In several embodiments, right ventricular pressure curves are derived from the TR curve, which can be utilized to identify physiological phases and/or calculated maximum and minimum pressure estimates.

In several embodiments, a TR Doppler signal may be interpolated. In some embodiments, TR Doppler signal interpolation is performed by cubic polynomial interpolation based on a polynomial of third-degree p(t), where t is the time normalized over the RR interval. In various embodiments, TR Doppler signal cubic polynomial interpolation is performed on one or more of the following phases of the TR signal: isovolumic phase, ejection phase, or the shoulder point. In several embodiments, utilizing a TR Doppler signal cubic polynomial interpolation result, right ventricular pressure is estimated. From the pressure signal, right ventricular systolic pressure is calculated. Utilizing interpolated signal can provide greater confidence in the estimation of pulmonary artery pressure. Further the interpolated signal can be utilized to assess the acquired TR Doppler signal quality and the TR Doppler signal tracing.

### BRIEF DESCRIPTION OF THE DRAWINGS

The description and claims will be more fully understood with reference to the following figures and data graphs, which are presented as exemplary embodiments of the disclosure and should not be construed as a complete recitation of the scope disclosed herein.

FIG. 1 provides a flow diagram of a method to estimate right ventricular pressure in accordance with various embodiments.

FIG. 2 provides a conceptual illustration of a computational processing system for analyzing sonographic data in accordance with various embodiments.

FIG. 3 provides schematics and data for the digitization of tricuspid regurgitation Doppler signals in accordance with various embodiments. Upper Left: Three cohorts were used for model derivation, validation, and clinical application (outlier cohort). Upper Right: The original recording was automatically cut into beats using ECG tracings, normalized to the cardiac cycle, and manually traced waveforms were extracted. Lower Left: Software extracted RVSP shows excellent agreement with reader derived values. Lower Right: Software extracted velocity time integral (VTI) shows excellent agreement with reader derived values.

FIGS. 4A, 4B, and 4C provide schematics and data for analysis of the tricuspid regurgitation Doppler waveform in accordance with various embodiments. FIG. 4A: Association of systolic pressures measured from right heart catheter (RHC) with echo measured RVSP in the derivation cohort. FIG. 4B: Waveform features. FIG. 4C: Waveform features and their relationship to clinical RV characteristics reported as correlation coefficients. All results presented are statistically significant (p<0.05).

FIG. 5 provides methods and results of mathematical interpolation of physiological time points in accordance with various embodiments. Left: Physiological time points defined using analysis of first and second derivative. Upper Right: Cubic polynomial interpolation based on the pressure and first pressure derivative at time point t_{A} and t_{B}. Lower Right: Example of interpolation of a TR waveform based on isovolumic phase (dashed), ejection phase (dotted), and knee point (line, grey) vs. expert reader tracing (line, black).

FIG. 6A provides interpolated right ventricular systolic pressure results for the derivation and validation cohorts in accordance with various embodiments. Upper: All interpolation methods show excellent correlation with echo measured RVSP expert readings in both the derivation as well as the validation cohort (all R²>0.91). Correlation with RHC was lower but remains very high (all R²>0.84). Lower Left: Data from the derivation cohort for the correlation between knee point interpolation systolic pressure and expert reader derived RVSP. Lower Right: Bland-Altman comparison of knee point interpolation pressure and RHC SPAP in derivation cohort.

FIG. 6B provides data results for the correlation of RVSP based on interpolation vs. mean pulmonary arterial pressure measured by RHC in accordance with various embodiments.

FIG. 7 provides data results using a correction factor for isovolumic and ejection phase in accordance with various embodiments. Upper Left: Correction factor for isovolumic phase based on the derivation and validation cohort. Upper Right: Correction factor for the ejection phase interpolation based on the derivation and validation cohort. Lower Left: Absolute corrected interpolation difference with RHC. Lower Right: Relative corrected interpolation difference with RHC.

FIGS. 8A and 8B provide results of testing of interpolation method in the outlier cohort of clinical readings in accordance with various embodiments. FIG. 8A: Violin plot comparing corrected interpolation differences across clinical report, knee point, and corrected isovolumic and ejection phases absolute (left) and relative (right). FIG. 8B Left: Underestimated RVSP was corrected by the knee interpolation in n=7. FIG. 8B Right: Overestimated RVSP was corrected by the knee interpolation in n=5.

FIG. 9 provides correlation of of interpolation method estimated pressure and echo measured RVSP in a non-PAH cohort in accordance with various embodiments. The method estimates RVSP well in both the subgroup of patients with and without PH.

### DETAILED DESCRIPTION

Turning now to the drawings and data, various methods and systems for noninvasive estimation of right ventricular pressure signals utilizing cubic polynomial interpolation of tricuspid regurgitation (TR) Doppler signal are described in accordance with various embodiments. In several embodiments, right ventricular pressure is estimated for the diagnosis of pulmonary hypertension. In many embodiments, estimation of right ventricular pressure utilizes TR echocardiography Doppler signal acquired via sonography. In several embodiments, TR Doppler signal is digitized, ventricular physiological phases are identified, and the digitized Doppler signal is interpolated utilizing the physiological phase points. In many embodiments, interpolated TR Doppler signal is utilized to estimate maximal velocity of the TR. In several embodiments, the estimated maximal velocity is utilized to calculate right ventricular systolic pressure. In some embodiments, the right ventricular pressure is calculated from the maximal velocity via the Bernoulli equation.

A goal in clinical assessment of PH utilizing medical imaging, such as sonographic Doppler echocardiography, is to provide reliable right ventricular systolic pressure calculation for proper diagnosis of PH. Prior methods for computing right ventricular systolic pressure from TR Doppler signal is highly variable. Because of the high variability, right heart catheterization is typically performed to confirm any PH diagnosis provided by Doppler echocardiography. Accordingly, it would be an advance in the art to provide a standardized mathematic based method that provides reliable, automatic reproducible and user-independent estimation of right ventricular pressure such that diagnosis of PH can be performed and minimize the need of the invasive right heart catheterization procedure.

### Noninvasive Determination of Pulmonary Arterial Pressure

Several embodiments are directed to estimating right ventricular pressure utilizing echocardiography Doppler signal of tricuspid regurgitation (TR). In many embodiments, a TR signal is acquired, digitized and interpolated. In several embodiments, the digitized TR signal is utilized to identify one or more ventricular physiological phases. Ventricular physiological phases to be identified include (but are not limited to) the isovolumic phase, the ejection phase, and the shoulder point. In many embodiments, the one or more identified ventricular physiological phases is used in the interpolation of the digitized TR signal. In several embodiments, an interpolated TR signal is utilized to estimate a maximal velocity. In many embodiments, an estimated maximal velocity is utilized to compute a right ventricular systolic pressure.

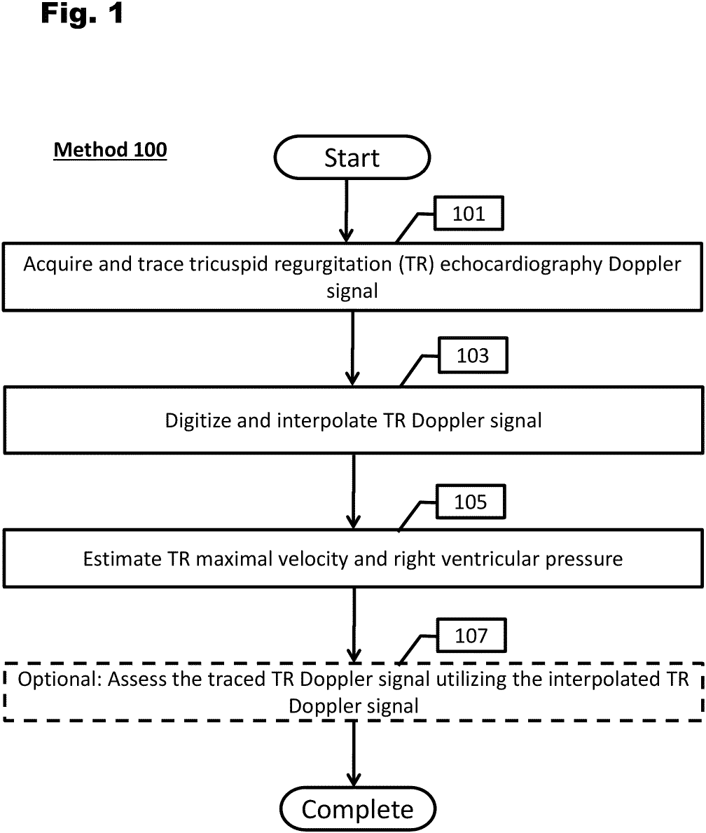

Provided in FIG. 1 is an exemplary method to estimate right ventricular pressure in accordance with various embodiments. Method 100 begins with acquiring (101) tricuspid regurgitation (TR) echocardiography Doppler signal. Doppler signals can be acquired utilizing sonography.

As depicted in FIG. 1, method 100 digitizes and interpolates (103) the acquired TR Doppler signal. To digitize the TR Doppler signal, in many embodiments, TR curves are traced, which can be performed manually or via automated software. In several embodiments, the traced TR curves are synchronized with electrocardiography and normalized to the RR interval. In some embodiments, the Doppler signal is filtered using a lowpass filter (e.g., 2nd-order Butterworth lowpass filter) with an appropriate cutoff (e.g., 10 Hz). Removal of low frequencies can help smooth the TR curves by removing spikes in frequency signal and/or smooth out ridges of the curve. From the TR curve, in accordance with many embodiments, the right ventricular pressure (RVP) is derived, which can be utilized to calculate right ventricular systolic/maximum pressure (RVSP), maximum and minimum pressure differentials (dP/dt max, dP/dt min), kurtosis and skewness. In various embodiments, maximum and minimum pressure differentials of the first and second pressure differentials are used to define one or more ventricular physiological phases, such as the isovolumic phase, the ejection phase, and the shoulder point. In some embodiments, first pressure derivatives are utilized to calculate dP/dt max and min to identify the isovolumic phase. In some embodiments, a second derivative is used to identify the beginning and end of the ejection phase as well as the shoulder point.

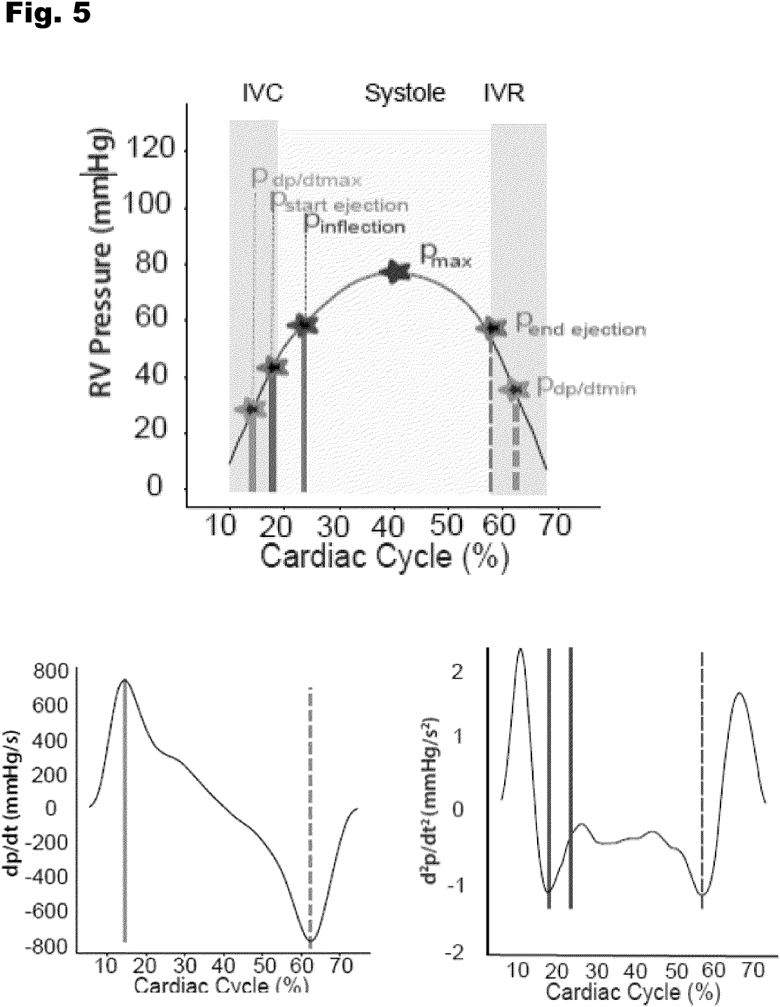

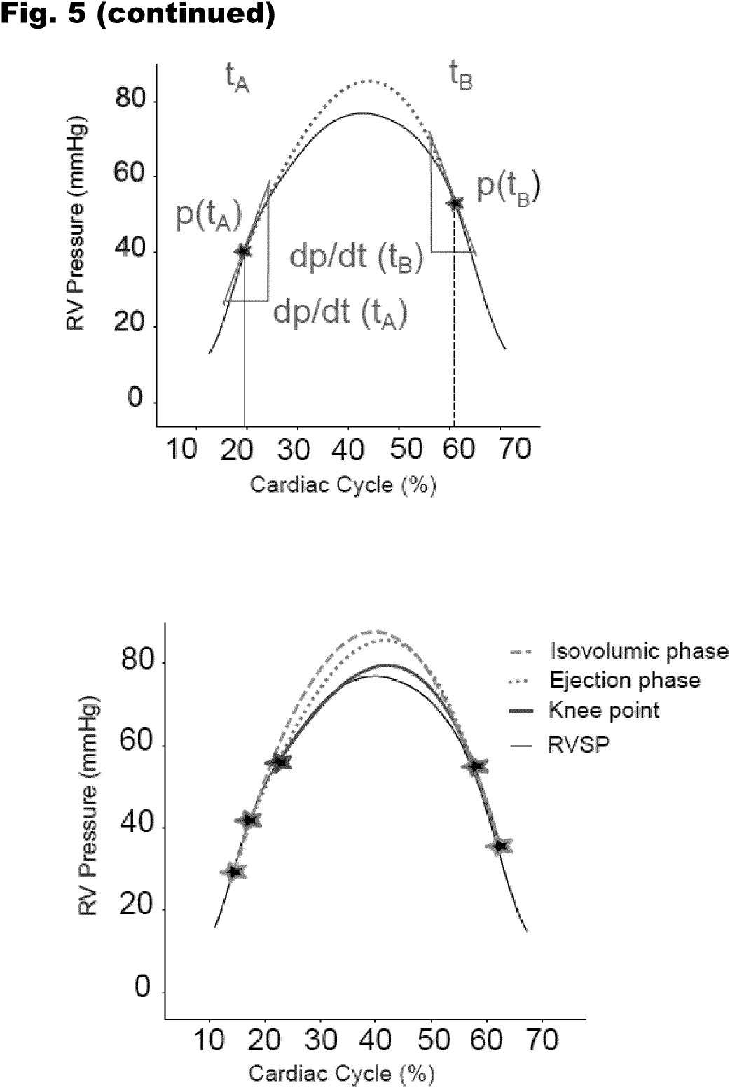

In several embodiments, cubic polynomial interpolation of the TR curve is performed. Alternatively, in some embodiments, cubic spline interpolation of the TR curve is performed. The decision to utilize cubic polynomial or cubic spline interpolation will depend on the desired outcome. To perform cubic polynomial interpolation, in accordance with many embodiments, the interpolation is based on a polynomial of third-degree p(t), where t is the time normalized over the RR interval. In many embodiments, parameters are calculated for each individual TR signal based on the boundary points of specific physiological phases and their respective local first derivatives, such that a smooth interpolated curve is generated. For more on parameter determination, see the Exemplary Embodiments and FIG. 5.

In many embodiments, isovolumic contraction phase starts at the beginning of the TR signals (isovolumic contraction (IVC)) and ends at the minima of the second derivative. Isovolumic relaxation (IVR) phase starts with the second minima of the second derivative and ends at the end of the Doppler signal. In several embodiments, the ejection phase starts and ends with the opening and the closing of the pulmonary valve, which are equivalent to first and last minima of the second derivative. In many embodiments, the shoulder point interpolation is established based on the observed decrease in TR slope shortly after the start of the ejection, evidenced in the curve shape visually as a ‘shoulder’ point, and mathematically by the constant second derivative. In some embodiments, the ‘shoulder’ point was mathematically defined as the second derivative of the TR velocity signal d²v/dt² < 500 m/s³ after the beginning of ejection. In various embodiments of shoulder point determination, a d²v/dt² < 500 m/s³, a d²v/dt² < 400 m/s³, a d²v/dt² < 300 m/s³, a d²v/dt² < 200 m/s³, a d²v/dt² < 100 m/s³, or any appropriate cutoff is utilized.

Method 100 further estimates (105) tricuspid regurgitation maximal velocity (Vmax) and right ventricular systolic pressure (RVSP) using the interpolated Doppler signal. In several embodiments, the interpolated Doppler signal provides a Vmax. In many embodiments, the tricuspid regurgitation maximal velocity is utilized to calculate right ventricular systolic pressure using Bernoulli’s equation or a modified version thereof. In some embodiments, the following modified Bernoulli’s equation is utilized:

$$\text{RVSP} \text{=} \text{4} \text{*} {\left(\text{Vmax}\right)}^{2} + \text{RAP}$$

where RVSP is the right ventricular systolic pressure, Vmax is the maximal velocity, and RAP is the right atrial pressure. In many embodiments, RAP is estimated, which can be done based on acquired Doppler imaging. For instance, RAP can be estimated by inferior vena cava and hepatic vein size and/or diameter.

In some embodiments, a correction factor is utilized to calculate RVSP. For instance, for calculation RVSP at the isovolumic phase, it is expected that the pressure will be overestimated due to the fact that the isovolumic phase interpolation is prior to pulmonary valve opening and therefore change in curvature. In some embodiments, a linear regression is utilized to determine a correction factor. For more on correction factors, see the Exemplary Embodiments.

Method 100 optionally assesses (107) the traced TR Doppler signal utilizing the interpolated TR Doppler signal. Interpolation curves can be utilized to assess, confirm, and/or verify the acquired TR Doppler signal and tracings of the signal, whether performed manually or via software. Interpolation curves and methods of their use can also be utilized to assess, confirm or verify automated Doppler methods used to calculate RVSP. Accordingly, in some embodiments, interpolation is performed to confirm and/or verify clinical estimates of RVSP or other systolic pressures.

While specific examples of methods for estimating right ventricular pressure utilizing echocardiographic Doppler signals are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for estimating right ventricular pressure appropriate to the requirements of a given application can be utilized in accordance with various embodiments of the invention.

Furthermore, estimated right ventricular systolic pressure can be utilized to diagnose pulmonary hypertension. When the pulmonary valve is open, right ventricular systolic pressure corresponds to the systolic pulmonary arterial pressure (sPAP) in the absence of outflow track obstruction. Once sPAP is estimated, it can be used to estimate mean pulmonary arterial pressure (mPAP). Several well-established equations can be utilized to estimate mPAP, such as the Chemla equation (see the Exemplary Embodiments). In accordance with generally accepted medical practices, a mPAP of greater than 20 mmHg is consistent with pulmonary hypertension.

### Applications Utilizing Estimated Right Ventricular Systolic Pressure

Various embodiments are directed to performing diagnostics and treatments upon individuals based on their estimated right ventricular systolic pressure as determined utilizing echocardiographic Doppler signal. As described herein, an individual may be determined as having an elevated mPAP and thus at risk of developing or has developed PH. In some embodiments, the estimated ventricular systolic pressure and/or a derivative thereof is utilized to perform a clinical intervention. Clinical interventions include performing a confirmatory diagnostic and/or administering a treatment.

### Diagnostic Methods

A number of embodiments are directed towards diagnosing an individual using estimated right ventricular systolic pressure and/or a derivative thereof. In some embodiments, a TR echocardiographic Doppler signal is acquired and traced; then digitized and interpolated to estimate right ventricular systolic pressure. In some embodiments, the estimated right ventricular systolic pressure is utilized to determine an individual’s mPAP, which can be further used to diagnose PH.

In several of embodiments, a diagnostic can be performed as follows:
perform echocardiography Doppler signal on the tricuspid valve
estimate right ventricular pressure based on a trace of tricuspid regurgitation
determine a pulmonary arterial pressure
diagnose the individual as at risk of developing or having developed pulmonary hypertension based on the pulmonary arterial pressure.

Diagnoses, in accordance with various embodiments, can be performed utilizing an estimated right ventricular pressure, which can be estimated as described in FIG. 1. In some embodiments, a PH diagnosis is based on a pulmonary arterial pressure being equal to and/or above a threshold. The threshold can be any appropriate diagnostic threshold, such as thresholds established by American Heart Association, American College of Cardiology, European Society of Cardiology, European Respiratory Society, or the World Health Organization. Although thresholds can vary between professional health organizations, common thresholds are mPAP ≥ 20 mmHg or mPAP ≥ 25 mmHg when the individual is at rest. Further, it is to be understood that professional health organizations can change the diagnostic threshold as appropriate.

### Medications, Supplements, and Further Diagnostics

Several embodiments are directed to the use of medications and/or dietary supplements to treat an individual in based upon an estimated right ventricular pressure and/or a derivative thereof. In some embodiments, medications and/or dietary supplements are administered in a therapeutically effective amount as part of a course of treatment. As used in this context, to “treat” means to ameliorate at least one symptom of the disorder to be treated or to provide a beneficial physiological effect. A therapeutically effective amount can be an amount sufficient to prevent reduce, ameliorate or eliminate symptoms of PH or pathological conditions related to PH, such as (for example) heart failure, arrhythmias, blood clots, bleeding in the lungs, or pregnancy complications.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result.

A number of medications and treatments are known for pulmonary hypertension and related disorders. Accordingly, embodiments are directed toward treating an individual with a treatment regimen when diagnosed with PH as described herein. Once diagnosed for PH, treatments include administration of a vasodilator (e.g., epoprostenol or treprostinil), administration of a guanylate cyclase stimulator (e.g., riociguat), administration of an endothelin receptor antagonist (e.g., bosentan, macitentan, or ambrisentan), administration of a phosphodiesterase 5 inhibitor (e.g., sildenafil or tadalafil), administration of a calcium channel blocker (e.g., amlodipine, diltiazem, or nifedipine), administration of an anticoagulant (e.g., warfarin), administration of a sodium-potassium ATPase pump inhibitor (e.g., digoxin), atrial septosmotomy, valve repair, and valve replacement.

### Computational Processing System

A computational processing system to estimate right ventricular pressure in accordance with various embodiments of the disclosure utilizes a processing system including one or more of a CPU, GPU and/or neural processing engine. In a number of embodiments, captured sonographic data is processed to generate a tricuspid regurgitation echocardiography Doppler signal using a computational processing system. In some embodiments, the computational processing system is housed within a sonographic imaging modality. In some embodiments, the computational processing system is housed separately from and receives the acquired sonographic data. In certain embodiments, the computational processing system is in communication with the imaging modality. In various embodiments, the processing system communicates with the imaging modality by any appropriate means (e.g., a wireless connection). In certain embodiments, the computational processing system is implemented as a software application on a computing device such as (but not limited to) mobile phone, a tablet computer, a wearable device (e.g., watch), and/or portable computer.

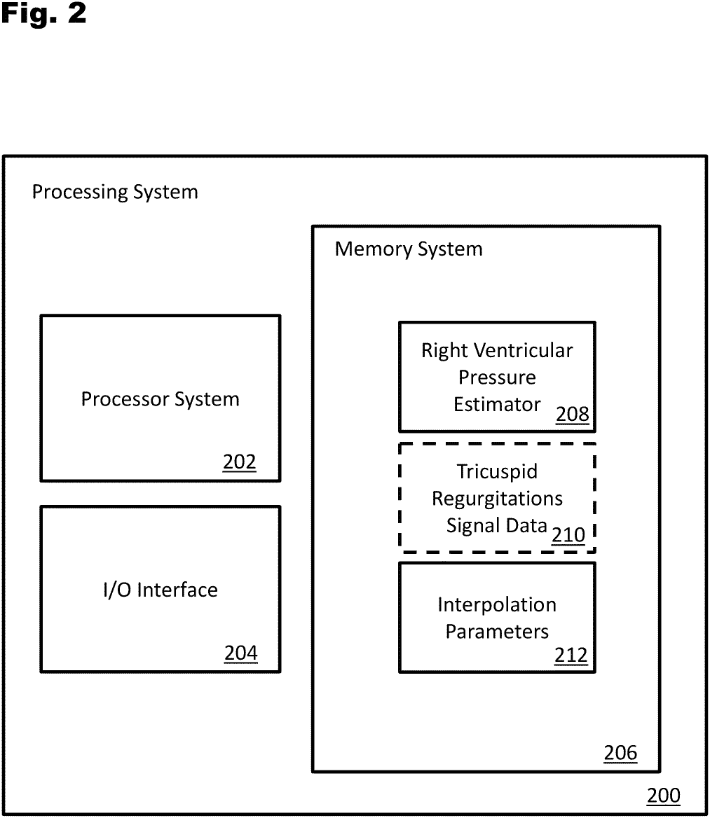

A computational processing system in accordance with various embodiments of the disclosure is illustrated in FIG. 2. The computational processing system 200 includes a processor system 202, an I/O interface 204, and a memory system 206. As can readily be appreciated, the processor system 202, I/O interface 204, and memory system 206 can be implemented using any of a variety of components appropriate to the requirements of specific applications including (but not limited to) CPUs, GPUs, ISPs, DSPs, wireless modems (e.g., WiFi, Bluetooth modems), serial interfaces, depth sensors, IMUs, pressure sensors, ultrasonic sensors, volatile memory (e.g., DRAM) and/or non-volatile memory (e.g., SRAM, and/or NAND Flash). In the illustrated embodiment, the memory system is capable of storing a right ventricular pressure estimator application 208. The right ventricular pressure estimator application can be downloaded and/or stored in non-volatile memory. When executed the right ventricular pressure estimator application is capable of configuring the processing system to implement computational processes including (but not limited to) the computational processes described above and/or combinations and/or modified versions of the computational processes described above. In several embodiments, the right ventricular pressure estimator application 208 utilizes tricuspid regurgitation signal data 210, which is optionally stored in the memory system, to perform signal processing including (but not limited to) digitizing tricuspid regurgitation signal data, interpolating tricuspid regurgitation signal data, and estimating maximal velocity. In certain embodiments, the digitizing tricuspid regurgitation signal data application 208 utilizes interpolation parameters 212 stored in memory to process acquired signal data to perform processes including (but not limited to) performing TR Doppler signal cubic polynomial interpolation on one or more of the following phases of the TR signal: isovolumic phase, ejection phase, or the shoulder point (also referred to as the knee point). In several embodiments, the tricuspid regurgitation signal data 210 is temporarily stored in the memory system during processing and/or saved for use in establishing interpolation parameters.

While specific computational processing systems are described above with reference to FIG. 2, it should be readily appreciated that computational processes and/or other processes utilized in the provision of right ventricular estimation in accordance with various embodiments of the disclosure can be implemented on any of a variety of processing devices including combinations of processing devices. Accordingly, computational devices in accordance with embodiments of the disclosure should be understood as not limited to specific imaging systems, computational processing systems, and/or right ventricular pressure estimator systems. Computational devices can be implemented using any of the combinations of systems described herein and/or modified versions of the systems described herein to perform the processes, combinations of processes, and/or modified versions of the processes described herein.

### EXEMPLARY EMBODIMENTS

The embodiments of the disclosure will be better understood with the various examples provided. Described herein are examples of estimating right ventricular systolic pressure utilizing echocardiographic Doppler signals. Results of estimated right ventricular systolic pressure are compared with standard practices within the field of cardiology. The results demonstrate the improvements provided by the embodiments described herein.

### Elucidating Tricuspid Doppler Signal Interpolation and Its Implication for Assessing Pulmonary Hypertension

This study had three main objectives to define the conceptual framework of TR waveform analysis. First, the TR waveform and its relationship with physiological metrics were analyzed, the metrics including but not limited to right ventricular (RV) longitudinal strain, TR severity, pulmonary vascular resistance (PVR), and heart rate. Second, a cubic polynomial interpolation method of estimating RVSP was derived and validated from different phases of the cardiac cycle. Finally, it was determined whether cubic polynomial interpolation could improve estimation of RVSP in cohort with greater bias between RVSP estimation from echo and RHC.

### Methods

First, a script for extraction of TR tracings was developed with automated analysis of normalized TR duration, skewness, kurtosis and maximal and minimal first pressure time derivatives (dp/dt max and min). Subsequently, RV pressure curves were constructed using the Bernoulli equation by adding estimated RAP. Then, a cubic polynomial interpolation model was derived to guide estimation of Vmax and RVSP.

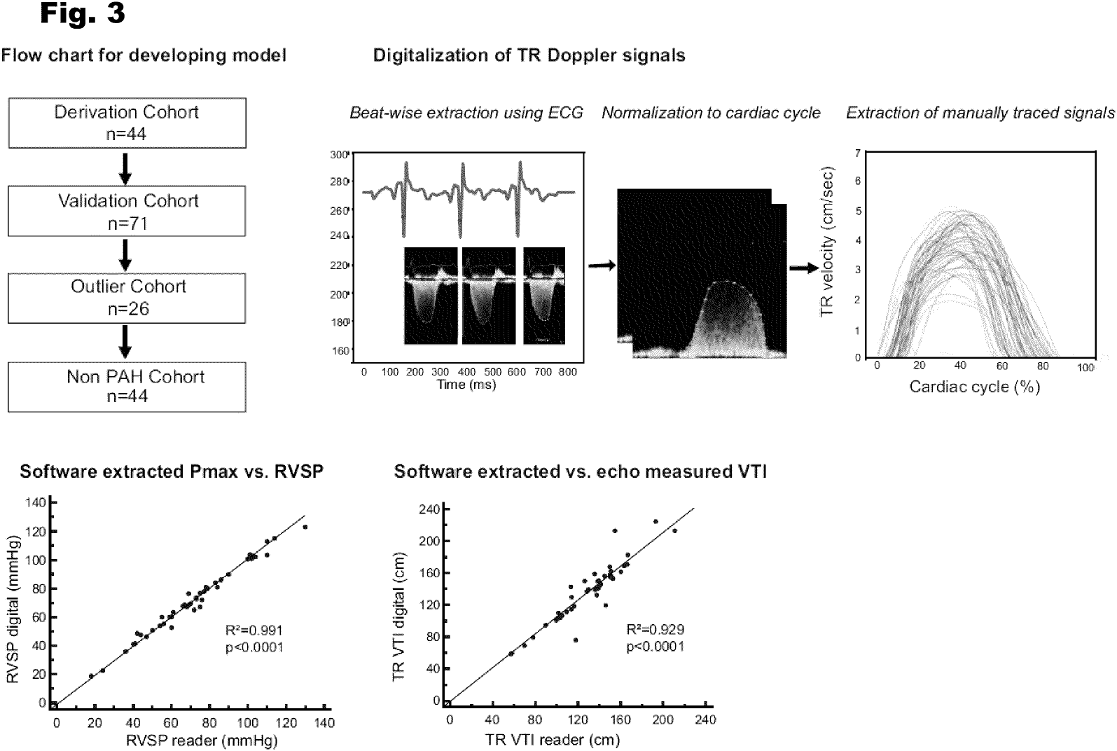

Clinical cohorts: Three cohorts were analyzed as part of this study: (1) a derivation cohort to evaluate cubic polynomial interpolation method, (2) a separate validation cohort, (3) a cohort to test the clinical applicability of the interpolation methods, (4) and a non-PAH cohort with and without PH (FIG. 3). The derivation cohort included 44 patients recruited between January 2007 and June 2009 with a diagnosis of pulmonary arterial hypertension (PAH) and in whom echo and right heart catheterization was obtained within 12 hours of each other in stable clinical condition. The validation cohort (n=71) patients were enrolled between December 2006 and December 2013, from the previously published prospective Vera Moulton Wall Center registry (M. Amsallen, et al, Circ Cardiovasc Imaging. 2017;10:e005771, the disclosure of which is herein incorporated by reference). A third “outlier” cohort of 22 patients with advanced lung disease and found to have a difference of greater than 30% between echocardiographic and invasive estimates. A non-PAH group (n=44) with referred to right heart catheterization was also added to assess performance of the method in another nosological context. The diagnosis of PAH was defined by the presence of mean pulmonary arterial pressure (mPAP) > 20 mmHg, pulmonary arterial wedge pressure ≤ 15 mmHg, and WHO group 1 diagnosis. All patients were in sinus rhythm at the time of echo, presence of right bundle branch block and QRS duration were recorded. Stanford University Institutional Review Board approved the study, which was conducted in agreement with the Helsinki-II declaration.

Echocardiography: Studies were acquired using Philips IE 33 ultrasound systems (Philips, Amsterdam, The Netherlands). All measurements were performed according to the latest guidelines by certified level 3 expert readers [C. V., M. A., F. H.] (see, R. M. Lang, et al., J Am Soc Echocardiogr. 2015;28:1-39; and S. E. Wiegers, et al., Circ Cardiovasc Imaging. 2019: 26; the disclosures of which are herein incorporated by reference). The reader only selected complete TR signals, for which the curve was interpretable for the entire cardiac cycle. Measures of RV size and function included relative right ventricular area (right to left area ratio), RV fractional area change and RV longitudinal strain. RV free wall Lagrangian longitudinal strain (RVLS) was measured from mid-endocardial end-diastolic and end-systolic manually traced lengths and calculated as: (end-systolic length - end-diastolic length. RAP was estimated from the inferior vena cava size and collapse according to American Society of Echocardiography guidelines (L. G. Rudski, et al., J Am Soc Echocardiogr. 2010;23:685-713, the disclosure of which is herein incorporated by reference).

Right heart catheterization: RHC was performed through the internal jugular or right femoral veins. Mixed venous saturation, RAP, systolic pulmonary artery pressure (SPAP), mean pulmonary arterial pressure (MPAP) diastolic pulmonary arterial pressure, and pulmonary capillary wedge pressure, were measured, and pulmonary vascular resistance (PVR), and cardiac index (using the assumed Fick method) subsequently calculated (see, C. G. LaFarge and O. S. Miettinen, Cardiovasc Res. 1970;4:23-30, the disclosure of which is herein incorporated by reference).

Digitization of TR signals using a novel semi-automated analysis: The outline of the TR Doppler waveform was first manually segmented then automatically extracted with the ECG and normalized to the R-R interval (FIG. 3). The TR waveform was filtered using a 2nd-order Butterworth lowpass filter using a 10 Hz cut-off. Digitization of the TR signals was reliable as assessed by the coefficient of determination for RVSP (R²=0.991) and velocity time integrals (VTIs) (R²=0.929) (FIG. 3). The analysis of the velocity profiles included the Vmax and the VTI, skewness and kurtosis. Skewness was defined as time to maximal pressure (Pmax) normalized by the duration of TR signal. RV pressure curves were constructed using the Bernoulli equation by adding estimated RAP. The first pressure derivatives were derived to calculate dP/dt max and min. The second derivative was used to identify the beginning and end of the ejection phase as previously described by Vanderpool et al (R. R. Vanderpool, et al., Pulm Circ. 2020;10:1-11, the disclosure of which is herein incorporated by reference).

Cubic polynomial interpolation: Polynomial interpolation is based on a polynomial of variable-degree p(t), where t is the time normalized over the RR interval. A second-degree polynomial interpolation would be synonymous with a parabolic fit of the TR signal, assumed in the guidelines (see, P. Lancellotti, et al., Eur J Echocardiogr. 2010;11:307-32, the disclosure of which is herein incorporated by reference). In contrast, a cubic polynomial approach was used, which additionally captures the skewness of the TR waveform. The four parameters were calculated for each individual TR waveform based on the boundary points of specific physiological phases and their respective local first derivatives, enforcing a smooth interpolated curve. Therefore, each patient’s curve interpolation was based on the TR curve only, not on an averaged interpolation from the entire cohort. The relevant physiological phases were found using the derivation cohort.

Various mathematical methods are commonly used for data interpolation: piecewise constant (nearest neighbor), linear, or polynomial functions of varying degree, where constant and linear functions are zero and first-degree polynomials, respectively. In polynomial interpolation, the interpolated section is approximated by a polynomial of degree n by fitting n+1 unknown coefficient based on n+1 datapoints. The cubic polynomial interpolation is based on a polynomial of third-degree p(t), where t is the time normalized over the RR interval.

$${p}_{T R} \left(t\right) = {a}_{1} + {a}_{2} ∗ t + {a}_{3} ∗ {t}^{2} + {a}_{4} ∗ {t}^{3}$$

The parameters of the cubic polynomial a₁ - a₄ are unknown and calculated for every TR waveform based on the continuity at the respective boundary points p(t₁) and p(t₂) including the smoothness condition on their derivative dp/dt (t₁) and dp/dt (t₂).

$$p \left({t}_{1}\right) = {a}_{1} + {a}_{2} ∗ {t}_{1} + {a}_{3} ∗ {t}_{1}^{2} + {a}_{4} ∗ {t}_{1}^{3}$$

$$p \left({t}_{2}\right) = {a}_{1} + {a}_{2} ∗ {t}_{2} + {a}_{3} ∗ {t}_{2}^{2} + {a}_{4} ∗ {t}_{2}^{3}$$

$$d p / d t \left({t}_{1}\right) = {a}_{2} + 2 {a}_{3} ∗ {t}_{1} + 3 {a}_{4} ∗ {t}_{1}^{2}$$

$$d p / d t \left({t}_{2}\right) = {a}_{2} + 2 {a}_{3} ∗ {t}_{2} + 3 {a}_{4} ∗ {t}_{2}^{2}$$

The above equations are written in matrix format with A consisting of the four unknown parameters a₁ - a₄, P as the fours pressure and pressure derivatives, and X including the time values. The linear system of equations is then solved for the unknown parameters a₁ - a₄ with Gauss elimination.

$$A = P / X$$

Statistical analyses: Continuous data are presented in terms of median and interquartile ranges and were compared between cohorts using the Mann-Whitney test, while Categorical data were presented as number and percentage and compared using Chi-square test between cohorts. Spearman correlation analysis was used to analyze association between variables and multivariable linear regression analysis to identify independent correlates. Bland-Altman analysis was also used to describe bias and limits of agreement between cubic polynomial interpolation and expert read echocardiographic studies and between echo and right heart catheterization measures. A paired t-test was used to analyze changes between clinical reports and cubic polynomial interpolation based on early phases of the TR signal. Results were considered significant when 2-sided p-values were <0.05. Analysis was performed using custom scripts in python (Python 3.0) with libraries PIL, scipy, and cv2 and using Medcalc statistical software (Version V19.8).

### Patient Sample Population

The characteristics of the derivation and validation cohort are presented in Table 1A, the characteristics of the non-PAH cohort in Table 1B. The mean MPAP, PVR, and RVLS in the validation cohort were 51 [47; 55] mmHg, 11.8 [1.4; 35.1] and -15.7[-16.8;-14.7], respectively. There were minor differences in cohort characteristics with small differences in RVLS and LVEF.

### TR Waveform Analysis Using Digital Extraction

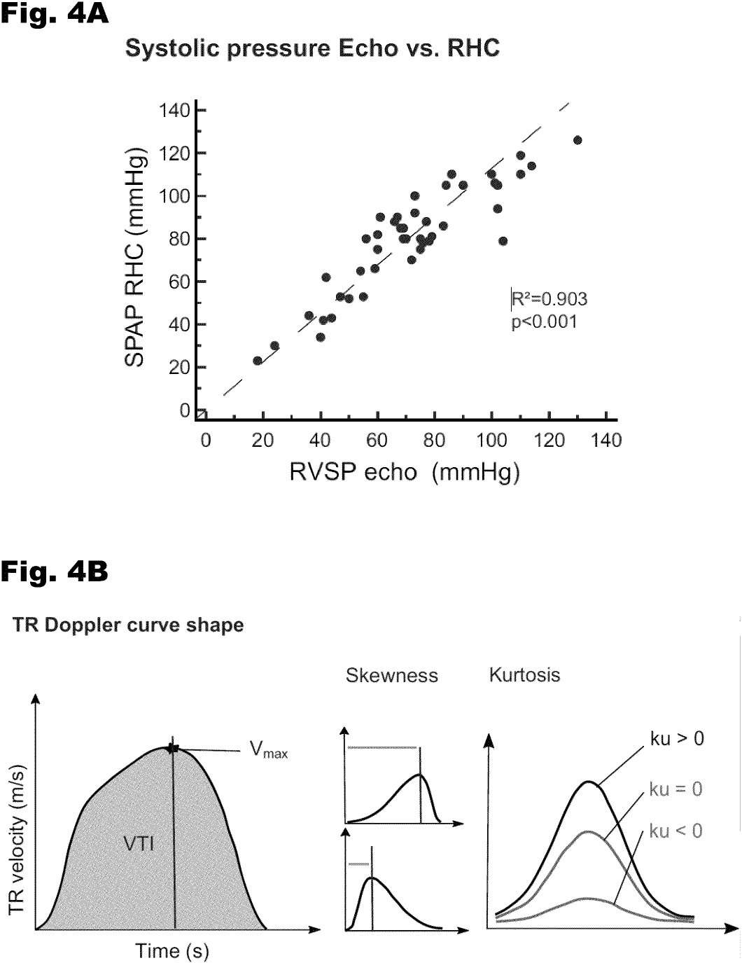

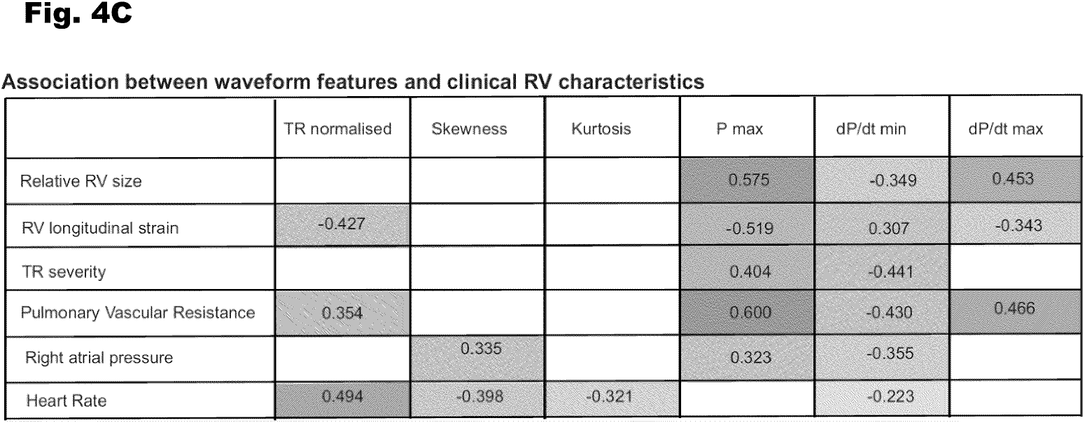

In the derivation cohort, the TR waveforms obtained were representative with excellent correlation between expert read echo and RHC (r = 0.90, p<0.001) and small negative bias -8.3 mmHg [-4.9; -11.6] (FIG. 4A). After digital extraction, normalized TR duration, skewness and kurtosis were analyzed (Table 2; FIG. 4B). As shown in FIG. 4C, the TR waveforms were non-parabolic with varying skewness. Normalized TR duration and corresponding (Bernoulli converted) peak pressure are the most closely related to right ventricular and pulmonary vascular characteristics. Normalized TR duration was moderately associated with RVLS, PVR, and heart rate; skewness was more strongly associated with RAP and heart rate, whereas Pmax was related to relative RV relative size, RVLS, RAP, PVR, and heart rate (FIG. 4C) (all p<0.001). Kurtosis on the other hand was only related to heart rate (p<0.001) (FIG. 4C). On multivariable analysis, Pmax was associated with RV size (p=0.0032) and PVR (p=0.0007), overall p<0.0001 and R²=0.64; while skewness was mainly related to TR normalized duration (p=0.0002, R² =0.60).

RVSP pressure curves was constructed based on the TR waveforms by adding estimated RAP. In the cohort, the estimated RAP was 10.1 mmHg [9.0;11.1] based on echo and 9.9 [8.7; 11.0] mmHg on RHC, with no significant difference (p=0.78), correlation R²=0.67, bias 0.2 mmHg.

For cubic polynomial interpolation, three different phases--the isovolumic, ejection and “knee” phase corresponding to an inflection point in the early ejection phase--were used (FIG. 5). These phases were identified using first and second derivatives of the digitized TR waveform. For ejection phase interpolation, the beginning of ejection was identified using the first second derivative minima corresponding to a change in the curvature associated with pulmonary valve opening. “Knee point” interpolation was based on the early inflection point after pulmonary valve opening. The “knee” point was mathematically defined as the second derivative of the TR velocity waveform d²v/dt² < 200 m/s³ after the beginning of ejection. The threshold was found iteratively in the derivation cohort and was applied to the validation cohort as well as the “outlier” cohort.

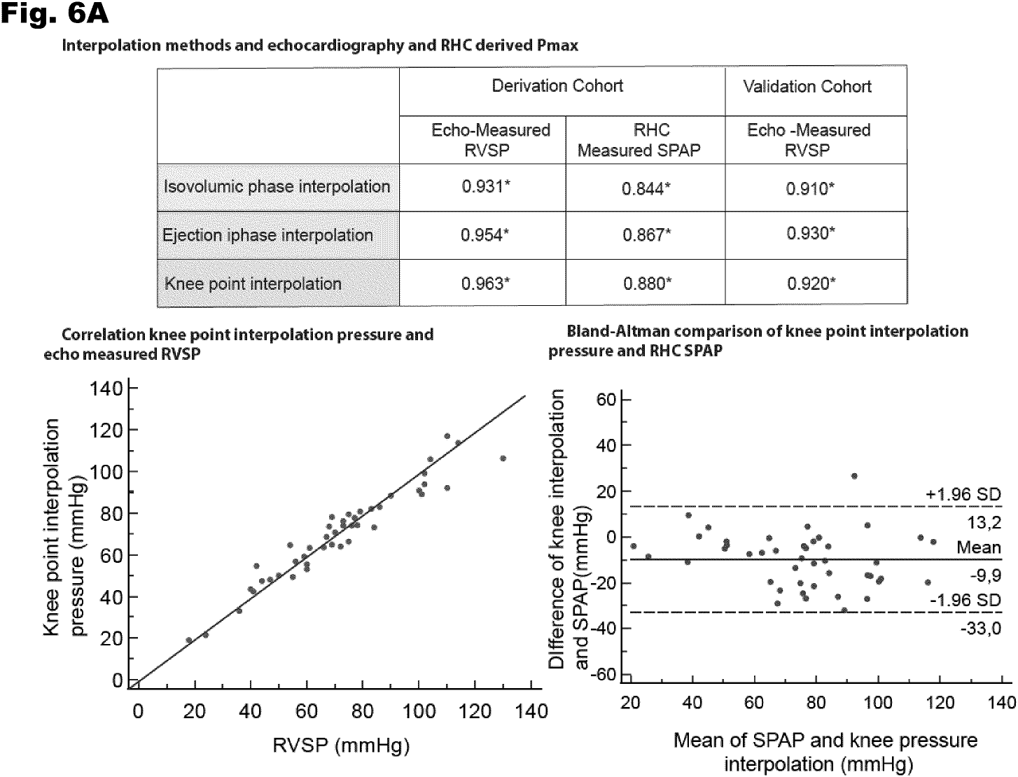

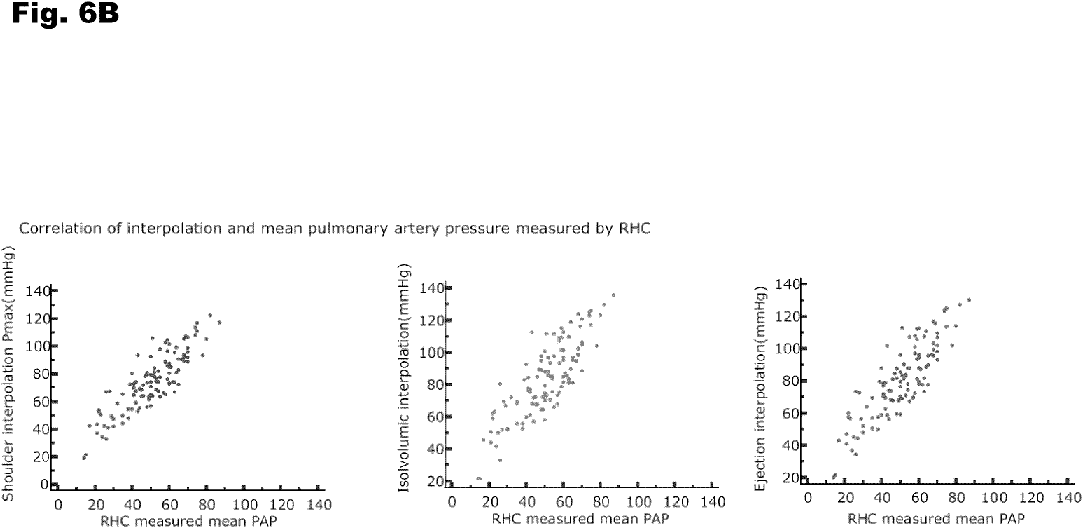

The cubic polynomial interpolation relied on the pressure at start time point t_{A} and end time point t_{B} defined in the physiological phases as well as the respective derivatives of the curve (FIG. 5). Local derivatives were calculated as a time-average over 3% of the cardiac cycle time, which was found to be optimal in the derivation cohort. FIG. 5 provides a typical example of cubic polynomial interpolation according to the three physiological phases. The three interpolation methods of Pmax were strongly associated with expert estimates of RVSP (all R²≥ 0.931) or SPAP or MPAP by RHC (all R² ≥ 0.844) (FIGS. 6A and 6B). While interpolation using the knee point provided a numerically higher coefficient of determination compared to the other methods of interpolation, the differences were not statistically different (p=0.54 for isovolumic phase, p=0.79 for ejection phase). The limits of agreement for isovolumic, ejection, and knee point interpolations were 6.03 [4.33; 7.73], -2.94 [1.47; 4.41], and -3.11 [-4.52; -1.71] mmHg, respectively (FIG. 6A).

The cubic polynomial interpolation method performed well in the validation cohort. The three methods had a high coefficient of determination when compared to expert reader (R² isovolumic=0.910, R² ejection phase=0.930, R² Knee point=0.920) (FIG. 6A).

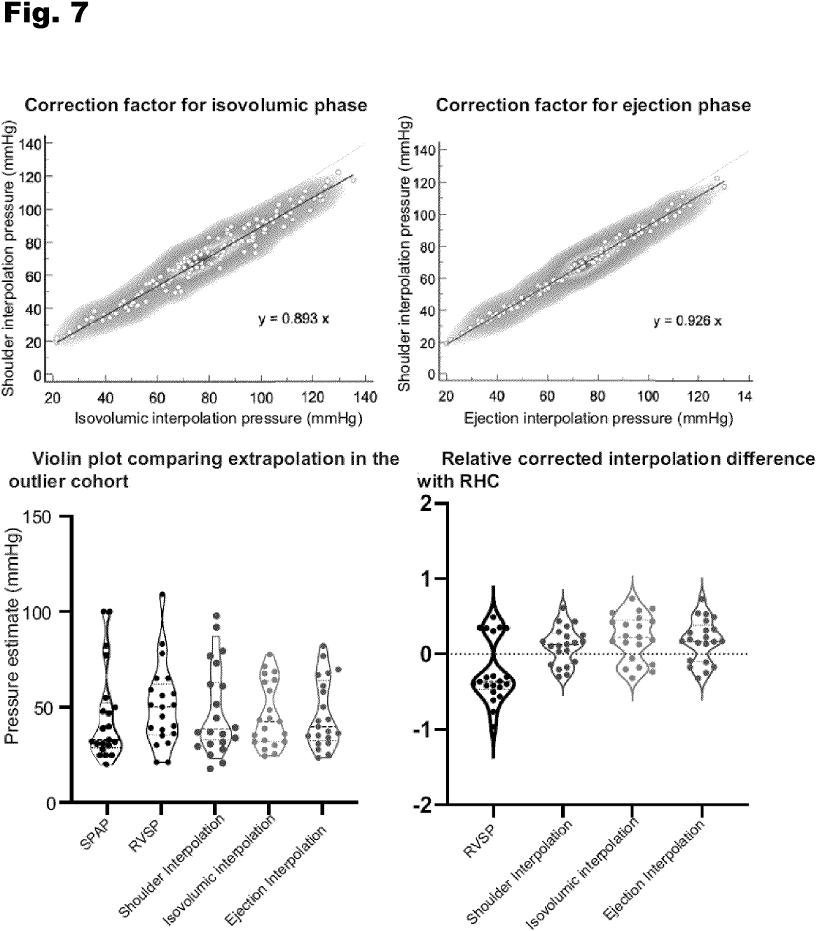

Since the isovolumic phase interpolation is prior to pulmonary valve opening and therefore change in curvature, it is expected that the pressure will be overestimated. Therefore, a correction factor needs to be incorporated and can be addressed using a fixed correction factor defined on the derivation and validation cohort with respect to the knee point. Using a linear regression equation, we found RVSP knee = 0.893 * RVSPiso (R²=0.98) and similarly RVSPₖₙₑₑ = 0.926 * RVSPₑⱼ (R²=1.0) (FIG. 7).

In the derivation and validation cohorts, severe TR was observed in 37% of patients. Pmax was statistically higher in patients with severe TR than in those with mild or moderate degree of TR (86+/-17 mmHg vs. 72+/- 24 mmHg, p=0.001). TR severity was not associated with the magnitude of difference between interpolation estimated RVSP and SPAP from RHC, regardless of the interpolation method used (isovolumic interpolation, 1.9+/-13.3 for non-severe TR vs. -0.19+/-15.11 for severe TR, p=0.43; knee interpolation, -6.95+/-11.9 vs. -9.75+/-13 mmHg, p=0.25; ejection interpolation, -1.49 vs. -2.64+/-14 mmHg, p=0.65).

Comparison with alternate linear regression model based on TR waveform parameters: As an alternative to cubic polynomial interpolation, we also tested whether linear regression based on RAP, pressure derivatives, and time intervals could provide estimates of peak signals. For RVSP estimates we included in the multivariable model normalized TR duration, dP/dt min and max, and RAP. Overall, the R² of the model was 0.78 (p<0.0001). When compared to the alternate multi-variable regression model, the cubic polynomial interpolation based on the knee performs better (R²=0.78 vs R²=0.94, p<0.0001). In the derivation cohort, variables retained were RVSP=0.038*dP/dt max -0.061 *dP/dtmin+ 0.96*RAP (R²=0.66, p<0.0001).

A correlation was seen between pressure at the start and end of RV ejection (Pₑⱼᵢ and Pₑⱼₑ), with MPAP and DPAP are respectively r=0.68 and r=0.75 for MPAP and r=0.69 and r=0.65 for DPAP. The R² was 0.83 for linear regression based on those two physiologically relevant points of the TR curve to predict Pmax (p<0.0001). The model was then defined as follows: Pmax=9.2+0.81*Pₑⱼₑ+0.45*Pₑⱼᵢ.

### Testing the Interpolation Method in the Outlier Cohort

The interpolation method was tested in the clinical setting using an outlier cohort with more than 30% relative difference between clinical echocardiographic and invasive measures. In the outlier cohort, echo estimates of RVSP was 50+/-21 mmHg, with mean RAP of 7 mmHg. The correction factor defined based on the validation and derivation cohorts was applied (uncorrected results presented in FIG. 7) to the outlier cohort to reduce overestimation of Pmax using interpolation from ejection and isovolumic phases.

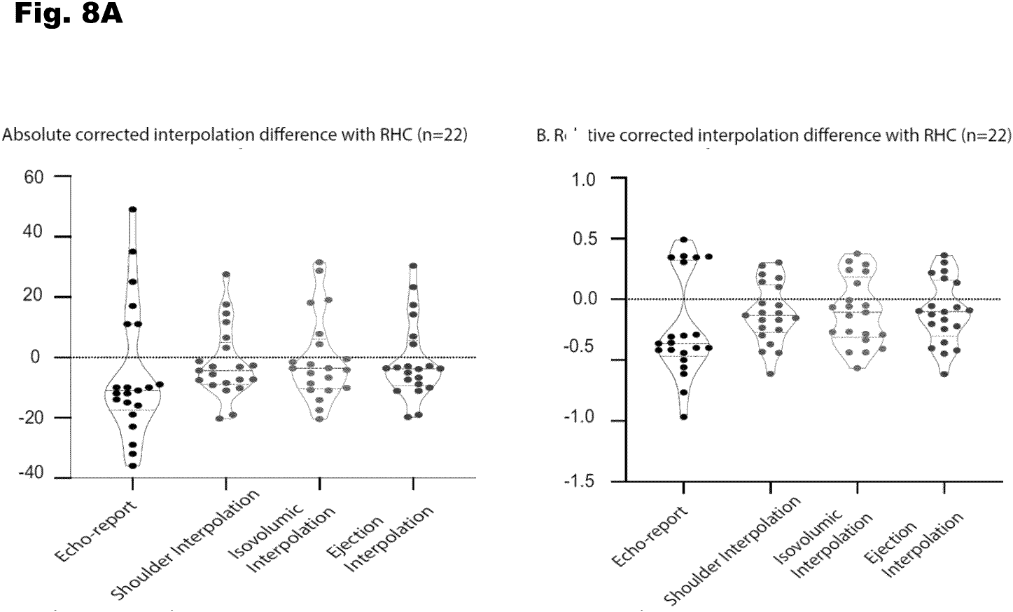

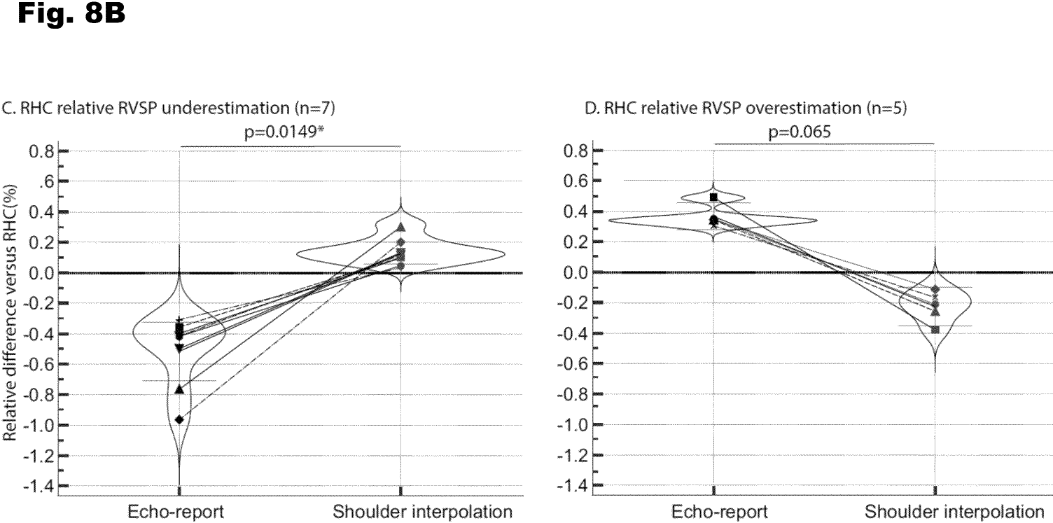

In the outlier cohort, the echo report results underestimated RVSP and showed high variability -11.0[-15.4;0.2] mmHg (FIG. 8A). Interpolation methods showed decreased variability with -4.4[-7.9;0.7] mmHg for knee interpolation, -3.2[-7.5;0.9] mmHg for ejection phase, -3.1[-8.6;2.0] mmHg for isovolumic phase interpolation (FIG. 8A). From the 22 patients in the outlier cohort, relative change in estimate from the knee interpolation and echo report of more than 20% was observed in 13 patients with 8 correcting an underestimation and 5 patients correcting an overestimation. When comparing to RHC, estimates by knee interpolation resulted in lower bias and smaller limits of agreement compared to the echo report (6 [1; 11] vs. 21 [12; 29] mm Hg, p=0.0149 for underestimation, -13.3 [-25.2;-1.3] vs. -26.2 [-46.42; -5.0] mm Hg, p=0.065 for overestimation) (FIG. 8B).

### Testing the Interpolation Method in the Non-PAH Cohort With or Without PH

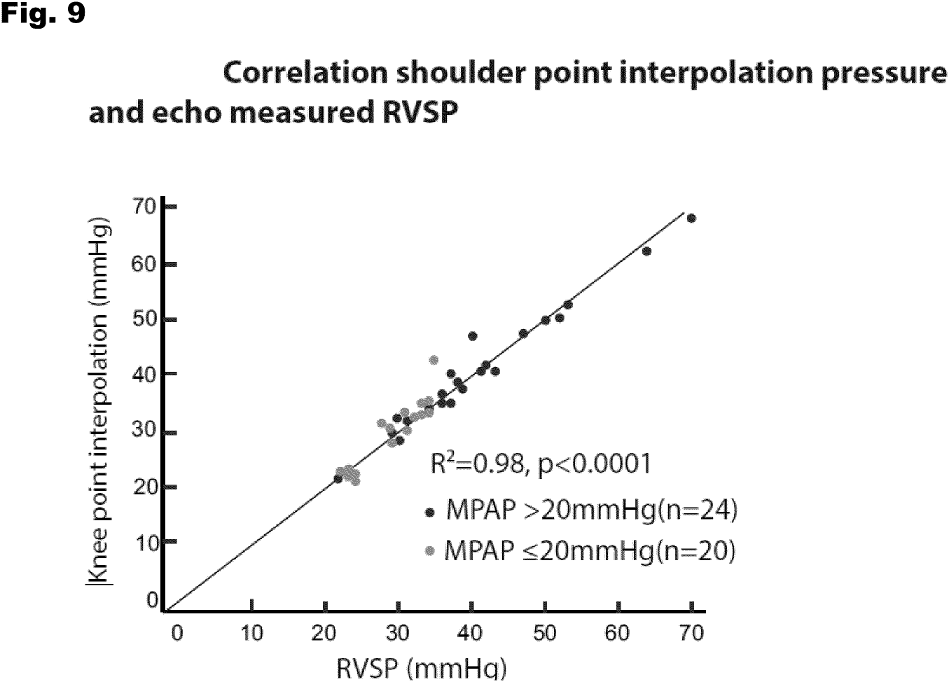

44 patients presenting left heart disease were selected as controls, 20 were without PH according to ESC guidelines with MPAP ≤ 20 mmHg based on right heart catheterization. Extrapolated Pmax from pressure at knee yielded a correlation of r=0.98 with RVSP as measured by echo. Correlation was not modified according to the presence or absence of PH with r=0.98 for patients with PH as previously defined vs. r=0.97 for patients without PH (FIG. 9).

When analyzing differences in TR waveform characteristics, it was observed that skewness of the TR waveform was higher in patients without PH compared to the pooled patients with PH from all cohorts 0.69 vs. 0.36, p<0.0001. Kurtosis of the TR waveform was similar in both groups 2.25 vs. 2.28, p=0.91.

In this study, a novel physiological approach to TR waveform analysis and interpolation was developed. The study had two main findings. First, it was found that the TR waveform was non-parabolic with significant variability in skewness. Second, cubic polynomial interpolation using isovolumic or early ejection phases (including knee) provided reliable interpolation of maximal RVSP. If further implemented, interpolation methods provide additional quality control for RVSP estimates to inform diagnosis of PH.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

TABLE 1A

Clinical cohorts main characteristics

| Variables | Derivation cohort (n=44) | Validation cohort (n=71) | p |
|---|---|---|---|
| Age, years | 48.6 [45.3; 51.9] | 48.6 [45.4; 51.9] | 0.07 |
| Female sex | 34 (74%) | 53 (74.6%) | 0.95 |
| Body surface area, m² | 1.85 [1.77;1.92] | 1.84 [1.79; 1.90] | 0.57 |
| Heart rate, bpm | 78 [73;83] | 83 [79; 86] | 0.91 |
| Hemodynamics | | | |
| Right atrial pressure, mmHg | 10 [8; 12] | 9 [8; 11] | 0.65 |
| Systolic pulmonary arterial pressure, mmHg | 80 [72; 87] | 83 [77; 89] | 0.81 |
| Mean pulmonary arterial pressure, mmHg | 49 [44; 54] | 51 [47; 55] | 0.66 |
| Pulmonary capillary wedge pressure, mmHg | 10 [8; 11] | 11 [9; 12] | 0.61 |
| Cardiac index, L/min/m² | 2.2 [2.0; 2.4] | 2.0 [1.8; 2.2] | 0.83 |
| Pulmonary vascular resistance, WU | 10.6 [8.9; 12.3] | 11.8 [1.4; 35.1] | 0.18 |
| Echocardiographic data | | | |
| Left ventricular ejection fraction, % | 56% [52; 61] | 60 [58; 63] | <0.001 |
| Left ventricular internal diameter, cm | 4.4 [4.2;4.6] | 4.1 [3.9; 4.3] | 0.93 |
| RVLS, % | -16.5[-18.7;14.3] | -15.7[-16.8;-14.7] | <0.0001 |
| RV end-systolic area index, cm²/m² | 13.4 [11.5; 15.2] | 15.8 [14.5; 17.1] | 0.82 |
| Tricuspid annular plane systolic excursion, cm | 1.9 [1.7; 2.1] | 1.5 [1.4; 1.7] | 0.41 |
| Tricuspid regurgitant severity>2,% | 11(25%) | 31(44%) | 0.04 |
| RVSP, mmHg | 72 [64; 77] | 81 [76; 86] | 0.38 |
| RA pressure, mmHg | 8 [6; 10] | 10 [9;12] | 0.08 |

TABLE 1B

Non-PAH cohort characteristics

| Variables | Non-PAH cohort (n=44) |
|---|---|
| Age, years | 60.9 [55.4; 66.4] |
| Female sex | 18 (41%) |
| Heart rate, bpm | 76 [70;82] |
| Hemodynamics | |
| Right atrial pressure, mmHg | 4 [3; 5] |
| Systolic pulmonary arterial pressure, mmHg | 33 [31; 36] |
| Mean pulmonary arterial pressure, mmHg | 21 [19; 23] |

TABLE 2

Curve parameters of the three cohorts

| Curve Parameters | Derivation cohort (n=44) | Validation cohort (n=71) | Outlier cohort (n=22) |
|---|---|---|---|
| Kurtosis | 2.33 [2.29;2.36] | 2.21 [2.18;2.25] | 2.16 [2.08;2.24] |
| Skewness | 0.68 [0.66;0.69] | 0.62 [0.59;0.65] | 0.67 [0.63;0.71] |
| dP/dt min | -582.6 [-623.6;-541.6] | -520.2 [-581.8;-458.6] | -360.6 [-425.5; -295.7] |
| dP/dtmax | 704.1 [649.5;758.7] | 555.7 [483.9;627.4] | 432.9 [352.4;513.5] |
| Normalized TR duration | 0.65 [0.63;0.68] | 0.65 [0.64;0.68] | 0.63 [0.56;0.69] |
| QRS duration, ms | 99 [95;104] | 103 [95;110] | 93 [84;105] |
| Right Bundle Branch Block, n (%) | 12 (27%) | 18 (25%) | 3 (13%) |

What is claimed is:

1. A method for determining right ventricular systolic pressure, comprising:
obtaining a sonographic Doppler signal of tricuspid regurgitation of an individual;
tracing a tricuspid regurgitation curve based on the sonographic Doppler signal;
identifying one or more ventricular phases;
interpolating the tricuspid regurgitation curve using cubic polynomial or cubic spline interpolation and the identified one or more ventricular phases to yield a maximal velocity; and
estimating right ventricular systolic pressure using the maximal velocity.

2. The method of claim 1, wherein the right ventricular systolic pressure is estimated using a Bernoulli’s equation or a modified Bernoulli’s equation.

3. The method of claim 2, wherein the right ventricular systolic pressure is estimated using the following modified Bernoulli’s equation:
$$\text{RVSP} = 4 * {\left(\text{Vmax}\right)}^{2} + \text{RAP}$$
where RVSP is right ventricular systolic pressure, Vmax is maximal velocity, and RAP is right atrial pressure.

4. The method of claim 1, wherein a correction factor is utilized to correct the estimated right ventricular systolic pressure.

5. The method of claim 4, wherein the correction factor is utilized to correct the estimated right ventricular systolic pressure when the ventricular phase is an isovolumic phase.

6. The method of claim 1 further comprising:
assessing the traced tricuspid regurgitation curve by comparing it to the interpolated tricuspid regurgitation curve.

7. The method of claim 1, wherein the one or more ventricular phases comprises the isovolumic phase, the ejection phase, or the shoulder point.

8. The method of claim 1, wherein the one or more ventricular phases are defined by a maximum and minimum pressure differential.

9. The method of claim 1, wherein the tricuspid regurgitation curve by a semi-automated analysis, comprising:
manually segmenting a waveform of the tricuspid regurgitation Doppler signal;
automatically extracting the waveform from the tricuspid regurgitation Doppler signal; and
normalizing the waveform of the tricuspid regurgitation Doppler signal to the R-R interval.

10. The method of claim 1 further comprising:
estimating a mean pulmonary arterial pressure from the estimated right ventricular systolic pressure.

11. The method of claim 10 further comprising:
diagnosing the individual as having pulmonary hypertension based on the estimated mean pulmonary arterial pressure.

12. The method of claim 11 further comprising:
administering a treatment to the individual for pulmonary hypertension.

13. A system for determining right ventricular systolic pressure, comprising:
a processor, a memory, and an application stored within the memory;
wherein the application directs the processor to:
acquire or receive a sonographic Doppler signal of tricuspid regurgitation of an individual;
trace or receive a tracing of a tricuspid regurgitation curve based on the sonographic Doppler signal;
identify one or more ventricular phases;
interpolate the tricuspid regurgitation curve using cubic polynomial or cubic spline interpolation and the identified one or more ventricular phases to yield a maximal velocity; and
estimate right ventricular systolic pressure using the maximal velocity.

14. The system of claim 13, wherein the processor is communication with a sonographic imaging modality, and wherein the imaging modality captures the sonographic Doppler signal.

15. The system of claim 14, wherein the processor and the memory is integrated within the sonographic imaging modality.

16. The system of claim 15, wherein the application directs the processor to acquire the sonographic Doppler signal.

17. The system of claim 14, wherein the processor and the memory is housed separately from the sonographic imaging modality.

18. The system of claim 13, wherein the application directs the processor to automatically trace the tricuspid regurgitation curve.

19. The system of claim 13, wherein the application directs the processor to assess the traced tricuspid regurgitation curve by comparing it to the interpolated tricuspid regurgitation curve.

20. The system of claim 13, wherein the application directs the processor to estimate a mean pulmonary arterial pressure from the estimated right ventricular systolic pressure.
